(19) United States
(12) Patent Application Publication
(10) Pub. No.: US 2022/0047492 A1
(43) Pub. Date: Feb. 17, 2022

(54) Multi-Benefit Personal Care Compositions and Methods for the Same

(71) Applicant: Colgate-Palmolive Company, New York, NY (US)

(72) Inventors: Shujiang CHENG, Warren, NJ (US); Qiang WU, Hillsborough, NJ (US); Adriana ARMAS, Ciudad de México (MX); Thomas BOYD, Metuchen, NJ (US)

(73) Assignee: Colgate-Palmolive Company, New York, NY (US)

(21) Appl. No.: 17/309,398

(22) PCT Filed: Nov. 27, 2018
(86) PCT No.: PCT/US2018/062620

§ 371 Date: May 25, 2021

(51) Int. Cl. A61K 8/73; A61Q 19/00; A61Q 17/00; A61Q 19/10

(52) U.S. Cl. CPC A61K 8/73; A61Q 19/10; A61Q 17/005; A61Q 19/007

(57) ABSTRACT

Personal care compositions and methods for increasing the production of natural moisturizing factors (NMFs) and increasing the production of antimicrobial peptides (AMPs) in skin are provided. The personal care compositions may include a carrier and an effective amount of one or more sulfated polysaccharides to increase NMFs and AMPs in skin when applied to the skin.

### BACKGROUND

Washing and cleansing skin with solid personal cleansing compositions may often leave the skin feeling dry. For example, conventional bar soaps may often include one or more surfactants, perfumes, preservatives, antimicrobial agents, and the like, that may strip moisture from the skin to thereby leave the skin feeling overly dry or chapped. In addition to over-drying skin, the increased use of conventional solid cleansing compositions that incorporate antimicrobial agents may lead to antimicrobial resistance.

In view of the foregoing, moisturizers (e.g., emollients) may often be added to the solid cleansing compositions and/or lotions may be applied direct to the skin after washing to replenish, condition, and/or prevent excess dryness of the skin. Additionally, antimicrobial agents may often be omitted or utilized sparingly in the solid personal cleansing compositions. While the moisturizers may attempt to restore or replenish some of the moisture stripped from the skin by conventional solid cleansing compositions, the moisturizers will not treat the skin by reversing the damage already caused by the conventional solid cleansing compositions. For example, the moisturizers included in the solid cleansing compositions do not promote or enhance the production of natural moisture factors (NMFs) in the skin to thereby treat or allow the skin to regulate its hydration and repair any damage. Additionally, the removal of antimicrobial agents from the cleansing compositions raises consumer concerns regarding the transmission of microbes.

What is needed, then, are improved solid personal cleansing compositions and methods for increasing the production of natural moisturizing factors and antimicrobial peptides in skin.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing a personal care composition comprising a carrier and one or more sulfated polysaccharides.

In at least one implementation, the one or more sulfated polysaccharides may be present in an effective amount to increase natural moisturizing factors (NMFs) in skin when applied to the skin.

In at least one implementation, the one or sulfated polysaccharides may be present in an effective amount to increase antimicrobial peptide (AMP) in skin when applied to the skin.

In at least one implementation, the one or more sulfated polysaccharides may be present in an amount of from greater than 0 weight % to about 5 weight %, from greater than 0 weight % to about 0.5 weight %, or from greater than 0 weight % to about 0.1 weight %, based on a total weight of the personal care composition.

In at least one implementation, the one or more sulfated polysaccharides may be present in an amount of greater than 0 weight %, greater than 0.05 weight %, greater than 0.1 weight or greater than 0.5 weight %, based on a total weight of the personal care composition.

In at least one implementation, the one or more sulfated polysaccharides may include a linear sulfated polysaccharide having one or more sulphate groups.

In at least one implementation, the one or more sulfated polysaccharides may include a natural sulfated polysaccharide.

In at least one implementation, the one or more sulfated polysaccharides may include a synthetic sulfated polysaccharide.

In at least one implementation, the one or more sulfated polysaccharides may include one or more of carrageenan, keratan sulfate, chondroitin sulfate, dextran sulfate, dermatan sulfate, fucoidan, funoran, heparin, porphyrin, or combinations thereof.

In at least one implementation, the one or more sulfated polysaccharides may include carrageenan, optionally, consists of carrageenan, further optionally, consists essentially of carrageenan.

In at least one implementation, the carrageenan may include one or more of kappa-carrageenan, iota-carrageenan, lambda-carrageenan, or combinations thereof, optionally, the carrageenan comprises, consists, or consists essentially of kappa-carrageenan.

In at least one implementation, the personal care composition consists essentially of the carrier and the one or more sulfated polysaccharides, optionally, the personal care composition consists of the carrier and the one or more sulfated polysaccharides.

In at least one implementation, the one or more sulfated polysaccharides consists essentially of carrageenan, optionally, the one or more sulfated polysaccharides consists of carrageenan.

In at least one implementation, the carrier is a solid carrier.

The foregoing and/or other aspects and utilities embodied in the present disclosure may be achieved by providing a method for preparing any one of the personal care composition disclosed herein. The method may include contacting one or more sulfated polysaccharides and a carrier with one another.

The foregoing and/or other aspects and utilities embodied in the present disclosure may be achieved by providing a method for treating, preventing, or reducing microbes on skin. The method may include contacting the skin with any one of the personal care compositions disclosed herein. The method may further include increasing production of antimicrobial peptides in or on the skin.

In at least one implementation, contacting the skin may increase production of antimicrobial peptide biomarker in or on the skin.

In at least one implementation, contacting the skin may increase or elicit an innate immune response.

The foregoing and/or other aspects and utilities embodied in the present disclosure may be achieved by providing a method for treating one or more dry skin conditions. The method may include contacting the skin with any one of the personal care compositions disclosed herein. The method may further include increasing an amount of natural moisturizing factors in or on the skin.

In at least one implementation, contacting the skin may increase an amount of Caspace-14 in or on the skin.

Further areas of applicability of the present disclosure will become apparent from the detailed description provided hereinafter, it should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

The present inventors have surprisingly and unexpectedly discovered that cleansing compositions, such as solid cleansing compositions, including one or more sulfated polysaccharides, such as carrageenan, increase the production of LL-37, which indicates the increase of antimicrobial peptide (AMP) production. The present inventors have also surprisingly and unexpectedly discovered that cleansing compositions, such as solid cleansing compositions, including one or more sulfated polysaccharides, such as carrageenan, increase the amount of natural moisturizing factors (NMFs) in the skin. The present inventors have further surprisingly and unexpectedly discovered that cleansing compositions including carrageenan in amounts greater than 0 wt % and less than 0.1 wt % exhibit greater NMF production than cleansing compositions without carrageenan and cleansing compositions including greater than 0.5 wt % carrageenan.

### COMPOSITIONS

Compositions disclosed herein may be or include a personal care product or a personal care composition thereof. For example, compositions disclosed herein may be a personal care composition, a personal care product, or form a portion of the personal care composition or the personal care product. in an exemplary implementation, the compositions disclosed herein may be personal care compositions including a carrier and one or more sulfated polysaccharides. As further described herein, the personal care compositions disclosed herein may be capable of or configured to facilitate, promote, enhance, or otherwise increase the production or formation of natural moisturizing factors (NMFs) in skin, thereby increasing hydration and barrier functions of the skin. As such, the personal care compositions disclosed herein may be utilized in the treatment of any one or more dry skin conditions. Illustrative dry skin conditions may be or include, but are not limited to, atopic dermatitis, rosacea, psoriasis, or the like, or any combination thereof, The personal care compositions disclosed herein may also be capable of or configured to facilitate, promote, enhance, or otherwise increase the production or formation of antimicrobial peptides (AMPs) in skin, thereby providing protection on surfaces of the skin against one or more microbes and/or multicellular organisms (e.g., bacteria, viruses, etc.). As such, it should be appreciated that the personal care compositions disclosed herein may be capable of or configured to concurrently facilitate, promote, enhance, or otherwise increase the production or formation of both natural moisturizing factors (NMFs) and antimicrobial peptides (AMPS) in skin to provide multiple benefits to the skin.

### Sulfated Polysaccharides

The personal care composition may include one or more sulfated polysaccharides and/or salts thereof. The sulfated polysaccharides may be linear sulfated polysaccharides having one or more sulphate groups. The sulfated polysaccharides may be natural sulfated polysaccharides, semi-synthetic sulfated polysaccharides, and/or synthetic sulfated polysaccharides. For example, the sulfated polysaccharides may be derived, extracted, or otherwise obtained from a natural source, such as from red algae Furcellaria lumbricalisc. In another example, the sulfated polysaccharides may be synthesized by any known process. For example, the sulfated polysaccharides may be synthesized by reacting neutral polysaccharides or natural sulfated polysaccharides with a sulfating reagent. Illustrative sulfated polysaccharides may include, but are not limited to, carrageenan, keratan sulfate, chondroitin sulfate, dextran sulfate, dermatan sulfate, fucoidan, funoran, heparin, porphyran, and the like, and combinations thereof. The sulfated polysaccharides may also include salts of the sulfated polysaccharides, such alkali metal salts or alkaline earth metal salts of the sulfated polysaccharides. Illustrative sulfated polysaccharides may also include those derived from or contained in DANAGEL™, GELCARIN®, ISAGEL™, LACTARIN®, LACTOGEL™, SEAGEL®, SEAKEM®, SEASPEN®, VISCARIN®, or the like, or any combination thereof, each of which include carrageenan (CAS #9000-07-1) and are commercially available from FMC BioPolymer Corp. of Philadelphia, Pa. In a preferred implementation, the sulfated polysaccharide includes one or more carrageenans. The carrageenan may be or include one or more sulfate groups per disaccharide. For example, the carrageenan may include one or more of the following: kappa-carrageenan (one sulfate group per disaccharide), iota-carrageenan (two sulfate groups per disaccharide), lambda-carrageenan (three sulfate groups per disaccharide), or any mixture or combination thereof. In a preferred implementation, the sulfated polysaccharide includes at least kappa-carrageenan.

In at least one implementation, the personal care composition may consist of or consist essentially of the carrier and the one or more sulfated polysaccharides capable of or configured to facilitate, promote, enhance, or otherwise increase the production or formation of natural moisturizing factors (NMFs) in skin. For example, the personal care composition may consist of or consist essentially of the carrier and the one or more sulfated polysaccharides, and may further exclude any other components or ingredients that are capable of or configured to facilitate, promote, enhance, or otherwise increase the production or formation of natural moisturizing factors (NMFs) in skin.

In at least one implementation, the personal care composition may consist of or consist essentially of the carrier and the one or more sulfated polysaccharides capable of or configured to facilitate, promote, enhance, or otherwise increase the production or formation of antimicrobial peptides (AMPs) in skin. For example, the personal care composition may consist of or consist essentially of the carrier and the one or more sulfated polysaccharides, and may further exclude any other components or ingredients that are capable of or configured to facilitate, promote, enhance, or otherwise increase the production or formation of antimicrobial peptides (AMPs) in skin.

The one or more sulfated polysaccharides may be present in the personal care composition in an effective amount or a therapeutically effective amount. As used herein, the expression or term “effective amount of one or more sulfated polysaccharides,” or the like, may refer to an amount of the one or more sulfated polysaccharides sufficient to elicit a response (e.g., biological, medical, etc.) of a tissue, system, animal, or human that is being sought. For example, the one or more sulfated polysaccharides may be present in the personal care composition in an effective amount to increase the production of NMFs and/or increase the production of antimicrobial peptides (AMPs) in skin.

In at least one implementation, the one or more sulfated polysaccharides may be present in the personal care composition in an amount of from greater than 0 weight % to about 5 weight %, based on a total weight of the personal care composition. For example, the one or more sulfated polysaccharides may be present in the personal care composition in an amount of from greater than 0 weight %, about 0.01 weight %, about 0.1 weight %, about 0.2 weight %, about 0.3 weight %, about 0.4 weight %, about 0.5 weight %, about 0.6 weight %, about 0.7 weight %, about 0.8 weight %, about 0.9 weight %, about 1 weight %, about 1.2 weight %, about 1.4 weight %, about 1.6 weight %, about 1.8 weight %, about 2 weight %, about 2.2 weight %, or about 2.4 weight % to about 2.6 weight %, about 2.8 weight %, about 3 weight %, about 3.2 weight %, about 3.4 weight %, about 3.6 weight %, about 3.8 weight %, about 4 weight %, about 4.1 weight %, about 4.2 weight %, about 4.3 weight %, about 4.4 weight %, about 4.5 weight %, about 4.6 weight %, about 4.7 weight %, about 4.8 weight %, about 4.9 weight %, or about 5 weight %, based on a total weight of the personal care composition. In another example, the one or more sulfated polysaccharides may be present in the personal care composition in an amount of from greater than 0 weight % to about 5 weight %, about 0.01 weight % to about 5 weight %, about 0.1 weight % to about 4.9 weight %, about 0.2 weight % to about 4.8 weight %, about 0.3 weight % to about 4.7 weight %, about 0.4 weight % to about 4.6 weight %, about 0.5 weight % to about 4.5 weight %, about 0.6 weight % to about 4.4 weight %, about 0.7 weight % to about 4.3 weight %, about 0.8 weight % to about 4.2 weight %, about 0.9 weight % to about 4.1 weight %, about 1 weight % to about 4 weight %, about 1.2 weight % to about 3.8 weight %, about 1.4 weight % to about 3.6 weight %, about 1.6 weight % to about 3.4 weight %, about 1.8 weight % to about 3.2 weight %, about 2 weight % to about 3 weight %, about 2.2 weight % to about 2.8 weight %, or about 2.4 weight % to about 2.6 weight %, based on a total weight of the personal care composition. In yet another example, the one or more sulfated polysaccharides may be present in the personal care composition in an amount of from greater than 0 weight %, greater than 0.01 weight %, greater than 0.1 weight %, greater than 0.2 weight %, greater than 0.3 weight %, greater than 0.4 weight %, greater than 0.5 weight %, greater than 0.6 greater than %, greater than 0.7 weight %, greater than 0.8 weight %, greater than 0.9 weight %, or greater than 1 weight %, based on a total weight of the personal care composition.

In at least one implementation, the one or more sulfated polysaccharides may be present in the personal care composition in an amount of from greater than 0 weight % and less than or equal to about 0.5 wt %, or more preferably less than or equal to about 0.1 wt %, based on a total weight of the personal care composition. In another implementation, the one or more sulfated polysaccharides may be present in the personal care composition in an amount of from greater than 0 weight % and less than or equal to about 0.1 weight %, less than or equal to about 0.09 weight %, less than or equal to about 0.08 weight %, less than or equal to about 0.07 weight %, less than or equal to about 0.06 weight %, less than or equal to about 0.05 weight %, less than or equal to about 0.04 weight %, less than or equal to about 0.03 weight %, less than or equal to about 0.02 weight %, based on a total weight of the personal care composition.

### Carrier or Excipients

The personal care composition may include the sulfated polysaccharides dispersed in, mixed with, dissolved in, combined with, or otherwise contacted with the carrier or one or more excipients. In at least one implementation, the carrier may be capable of or configured to store, entrain, or otherwise contain the sulfated polysaccharides, and deliver the sulfated. polysaccharides to one or more tissues, such as skin. It should be appreciated that the components or contents of the carrier and the respective amount of each of the components of the carrier may be at least partially determined by the type or use of the personal care product or the composition thereof. Illustrative personal care products or compositions thereof that may include the sulfated polysaccharides may include, but are not limited to, antiperspirants, deodorants, body washes, shower gels, soaps, including bar soaps and liquid soaps (e.g., liquid hand soaps), face washes, shampoos, hair conditioners, lotions, moisturizers, serums, spot treatments, cosmetics, or the like. In a preferred implementation, the personal care product or the composition thereof that includes the sulfated polysaccharides are solid cleansing compositions, such as bar soaps.

In at least one implementation, the personal care product or the composition thereof may be a skin care product. Illustrative skin care product may be or include, but are not limited to, a lotion, a cosmetic, a sunscreen, or the like. The carrier of the skin care product may include, but is not limited to, any one or more of surfactants, conditioning agents, moisturizers, sunscreens, UV absorbers, antioxidants, enzymes and/or other proteins, vitamins, antibacterial agents, odor reducing agents, steroids, anti-inflammatory agents, naturally and/or non-naturally occurring humectants, skin lipid fluidizers, occlusive agents, amino acids, physical and/or chemical exfoliants, skin whiteners, anti-aging, antiperspirant actives, or the like, or any combination thereof.

In at least one implementation, the personal care product or the composition thereof may be a personal hand and/or body cleansing composition or a personal hand and/or body conditioning composition. Illustrative personal hand and/or body cleansing or conditioning compositions may include, but are not limited to, liquid soaps, bar soaps, body washes, shower gels, lotions, and the like. In a preferred implementation, the personal hand and/or body cleansing or conditioning composition is a solid personal hand and/or solid body cleansing or conditioning composition, such as bar soap. The carrier for the personal hand and/or body cleansing composition or the personal hand and/or body conditioning composition may include, but is not limited to, any one or more of fragrances, essential oils, emulsifying agents, thickening agents, colorants, surfactants, natural actives, therapeutic actives, stain prevention actives, antimicrobial agents, vitamins, natural extracts, amino acids, enzymes and/or other proteins, abrasives, odor control agents, conditioning agents, moisturizers, humectants, occlusive agents, skin lipid fluidizers, lipophilic actives, hydrophilic materials, pearlizers, opacifying agents, sodium soaps, titanium dioxide, fragrances, or the like, or any mixture or combination thereof, in addition to any one or more of the other carrier components as discussed above.

The carrier may be hydrophilic or hydrophobic. The carrier may be anhydrous. The carrier may be a liquid or a solid at room temperature. The carrier may have a viscosity of from about 2,000 centipoise (cP) to about 100,000 cP. For example, the carrier for a shower gel may have a viscosity of from about 2,000 cP to about 16,000 cP. In another example, the carrier for a lotion may have a viscosity of from about 10,000 cP to about 100,000 cP. Accordingly, it should be appreciated that the viscosity of the carrier may vary and may at least partially depend on the type of personal care composition. In an exemplary implementation, the carrier is a solid at room temperature.

Unless otherwise specifically identified, the ingredients for use in the compositions and formulations of the compositions disclosed herein are preferably cosmetically acceptable ingredients. As used herein, the expression “cosmetically acceptable” may refer to a component or ingredient that is suitable for use in a formulation for topical application to human skin. A cosmetically acceptable excipient, may refer to an excipient that is suitable for external application in the amounts and concentrations contemplated in the formulations of the compositions disclosed herein, and includes for example, excipients which are “Generally Recognized as Safe” (GRAS) by the United States Food and. Drug Administration (USDA).

### METHODS

The present disclosure may provide methods for preparing a personal care product or a personal care composition thereof. In at least one implementation, the method may include mixing, stirring, combining, or otherwise contacting a carrier and one or more sulfated polysaccharides with one another. In another implementation, the method may include adding, mixing, stirring, combining, or otherwise contacting one or more sulfated polysaccharides with a carrier. In at least one example, the carrier is a base solid cleansing composition. For example, the carrier may be a bar soap.

The present disclosure may also provide methods for treating microbes on skin and/or provide protection on surfaces of the skin from microbes. The method may include increasing production of AMPs in and/or on the skin by contacting any one or more of the personal care compositions disclosed herein with the skin. The method may also include reducing the amount of microbes on the skin by increasing the production of AMPs in the skin. The method may also include increasing production of AMP biomarker LL-37 and/or cathelicidins in and/or on the skin. The method for treating microbes on skin may also include enhancing or increasing the innate immune response by treating the skin with any one or more of the personal care compositions disclosed herein. The one or more microbes may be or include, but are not limited to, gram negative bacteria, gram positive bacteria, any strains that are resistant to conventional antibiotics, mycobacteria, enveloped viruses, fungi, transformed and/or cancerous cells, other microbes, or the like, or any combination thereof.

The present disclosure may further provide methods for treating or reducing one or more skin conditions, such as dry skin conditions. Illustrative dry skin conditions may be or include, but are not limited to, atopic dermatitis, rosacea, psoriasis, or the like. It should be appreciated that atopic dermatitis may result in a deficiency of filaggrin (filament-aggregating protein), a protein at least partially responsible for skin barrier functions and NMF production. It should further be appreciated that filaggrin contributes to the physical strength of the stratum corneum (SC) barrier through its integral involvement in the filament matrix complex in the inner layer of the stratum corneum (SC). In the outer layer of the SC, filaggrin is degraded into NMFs. It should be appreciated that NMFs are integral to the function of the SC as NMFs provide moisture retention (humectancy), maintain the acidic pH and buffering capacity of the SC, promote proper epidermal maturation and desquamation, and decrease pathogenic bacterial colonization. The method for treating or reducing one or more skin conditions may include increasing amounts of natural moisturizing factors (NMFs) in and/or on the skin by contacting any one or more of the personal care compositions disclosed herein with the skin. The method may include increasing production or amounts of a Caspace-14 gene in and/or on the skin to promote filaggrin degradation to thereby product NMF in and/or on the skin.

The present disclosure may also provide methods of cleansing skin and/or enhancing hydration and barrier functions of the skin in a patient in need thereof via the enhanced production of Caspase-14 and/or NMF. Patients in need thereof may have relatively lower natural moisturizing factors (NMFs) and/or relatively low amounts of concentration of Caspase-14 in the skin, which may be evidenced by dry and/or chapped skin. The method may include providing an effective amount of the personal cleansing composition to enhance Caspase-14 and/or NMF in skin, contacting the personal cleansing composition to the skin or hair, and optionally, rinsing the personal cleansing composition from the skin or hair with water. In at least one implementation, the personal cleansing composition may be combined with added water prior to or while contacting the personal cleansing composition with the skin or hair.

The present disclosure may also provide a personal care composition including a carrier and one or more sulfated polysaccharides for use in treating microbes on skin.

The present disclosure may also provide a personal care composition including a carrier and one or more sulfated polysaccharides for use in treating one or more dry skin conditions.

The present disclosure may further provide a method of making a personal care composition for treating microbes on skin and/or treating one or more dry skin conditions. The method may include combining or otherwise contacting one or more sulfated polysaccharides with a carrier to prepare the personal care composition.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

A control solid cleansing composition (1) and three test solid cleansing compositions (2)-(4) were prepared by adding a base cleansing composition including soap chips, titanium dioxide, and fragrance, with varying amounts of carrageenan according to Table 1. The carrageenan was obtained from FMC BioPolymer Corp. of Philadelphia, Pa. Each of the solid cleansing compositions (1)-(4) was tested as 1% soap samples. Particularly, each of the solid cleansing compositions (1)-(4) was diluted in water in a weight ratio of 1:20, and subsequently diluted in phosphate-buffered saline (PBS) in a weight ratio of 1:5.

TABLE 1
Compositions of Control and Test Solid
Cleansing Compositions (1)-(4)

| | (1) | (2) | (3) | (4) |
|---|---|---|---|---|
| | Amount | Amount | Amount | Amount |
| Ingredient | (wt %) | (wt %) | (wt %) | (wt %) |
| Soap Chips | 98.14 | 98.09 | 98.04 | 97.64 |
| Titanium dioxide | 0.46 | 0.46 | 0.46 | 0.46 |
| Fragrance | 1.4 | 1.4 | 1.4 | 1.4 |
| Carrageenan | 0 | 0.05 | 0.1 | 0.5 |
| Total | 100 | 100 | 100 | 100 |

### Example 2

Each of the control and test solid cleansing compositions (1)-(4) was evaluated in vitro on skin tissue models to observe the production of antimicrobial peptides (AMP), particularly, AMP LL-37. 3D human skin models obtained from MatTek Corp. of Ashland, Mass., were utilized as the models in the in vitro study, and LL-37 production was monitored with an ELISA Kit. To conduct the in vitro study, 30 μm of respective 1% solutions of the control solid cleansing composition (1) or one of the test solid cleansing compositions (2)-(4) were topically applied to respective 3D human skin models and incubated at about 37° C. for about 1 hour, After about 1 hour, each of the 3D human skin models were thoroughly and gently washed with PBS about 8 times. Each of the 3D human skin models was then placed in fresh media and incubated at about 37° C. for about 24 hours. The 3D human skin models were then collected and lysed four times with lysis buffer at 15.01/s for 15 minutes. After lysing with the lysis buffer, each of the lysed samples was frozen or maintained at about −80° C. The production of AMP biomarker LL-37, as measured by the ELISA kit, from respective human skin models treated with each of the control and test solid cleansing compositions (1)-(4) is summarized in Table 2. All measurements were done in triplicate, averaged, and normalized to the total protein in each of the 3D human skin models.

TABLE 2
Amount of LL-37 Measured from Skin
Models Treated with 1% Solutions of Control
and Test Solid Cleansing Compositions (1)-(4)

| | Amount of | Amount | | Change |
|---|---|---|---|---|
| | Carrageenan | of | Std | from |
| # | (wt %) | LL-37 | Dev | Control |
| 1 | 0 | 0.7 | 0.2 | — |
| 2 | 0.05 | 1.2 | 0.4 | 0.5 |
| 3 | 0.1 | 1.5 | 1.0 | 0.8 |
| 4 | 0.5 | 2.7 | 0.2 | 2.0 |

As illustrated in Table 2, increasing the concentration of carrageenan in the solid cleansing compositions (2)-(4) relative to the control solid cleansing composition (1), which did not include carrageenan, surprisingly and unexpectedly resulted in a corresponding increase in the amount of LL-37 measured from the 3D skin models. It should be appreciated that the increase in biomarker LL-37 indicates the increase of antimicrobial peptide (AMP) production, which was surprising and unexpected.

### Example 3

The control solid cleansing compositions (1) and three test solid cleansing compositions (2)-(4) prepared in Example 1 were evaluated in vitro on skin tissue models to observe the production of natural moisturizing factors (NMFs), particularly, Caspase-14. Each of the solid cleansing compositions (1)-(4) was tested as 1% soap samples. Particularly, each of the solid cleansing compositions (1)-(4) was diluted in water in a weight ratio of 1:20, and subsequently diluted in phosphate-buffered saline (PBS) in a weight ratio of 1:5. 3D human skin models obtained from MatTek Corp. of Ashland, Mass., were utilized as the models in the in vitro study, and the Caspase-14 production was monitored with an ELISA Kit.

To conduct the in vitro study, 30 μm of respective 2% solutions of the control solid cleansing composition (1) or one of the test solid cleansing compositions (2)-(4) were topically applied to respective 3D human skin models and incubated at about 37° C. for about 1 hour. After about 1 hour, each of the 3D human skin models was thoroughly and gently washed with PBS about 5 to about 8 times. Each of the 3D human skin models was then placed in fresh media and incubated at about 37° C. for about 24 hours. The 3D human skin models were then collected and lysed four times with lysis buffer at 15.01/s for 15 minutes. After lysing with the lysis buffer, each of the lysed samples was frozen or maintained at about −80° C. The production of NMF biomarker Caspase-14, as measured by the ELISA kit, from respective human skin models treated with each of the control and test liquid cleansing compositions (1)-(4) is summarized in Table 3. All measurements were done in triplicate and averaged unless indicated otherwise. All measurements were normalized to the total protein in each of the 3D human skin models.

TABLE 3
Amount of Caspase-14 Measured from Skin
Models Treated with 2% Solutions of Control
and Test Liquid Cleansing Compositions (1)-(4)

| | Amount of | | | |
|---|---|---|---|---|
| | Carrageenan | Amount of | | Change from |
| # | (wt %) | Caspase-14 | Std Dev | Control |
| 1 | 0 | 538 | 97 | — |
| 2 | 0.05 | 1223 | 114 | 685 |
| 3 | 0.1 | 668* | N/A* | 130 |
| 4 | 0.5 | 663* | N/A* | 125 |

*Only one tissue sample was evaluated.

As illustrated in Table 3, increasing the concentration of carrageenan in the solid cleansing compositions (2)-(4) relative to the control solid cleansing composition (1), which did not include carrageenan, result in a corresponding increase in the amount of Caspace-14. It should be appreciated that the increased amount of biomarker Caspase-14 indicates the production of NMF, as Caspace-14 is utilized in the degradation of filaggrin to produce NMF in skin. It was further surprisingly and unexpectedly discovered that carrageenan in amounts greater than 0 wt %, as provided in the control solid cleansing composition (1) and less than 0.1 wt %, as provided by the test solid cleansing composition (3) exhibited greater Caspace-14 than the control (1) and the solid cleansing composition (4), which had 0.5 wt % carrageenan.

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

1. A personal care composition, comprising a carrier and one or more sulfated polysaccharides.

2. The personal care composition of claim 1, wherein the one or more sulfated polysaccharides are present in an effective amount to increase natural moisturizing factors (NMFs) in skin when applied to the skin.

3. The personal care composition of claim 1, wherein the one or sulfated polysaccharides are present in an effective amount to increase antimicrobial peptide (AMP) in skin when applied to the skin.

4. The personal care composition of claim 1, wherein the one or more sulfated polysaccharides are present in an amount of from greater than 0 weight % to about 5 weight %, from greater than 0 weight % to about 0.5 weight %, or from greater than 0 weight % to about 0.1 weight %, based on a total weight of the personal care composition.

5. The personal care composition of claim 1, wherein the one or more sulfated polysaccharides are present in an amount of greater than 0 weight %, greater than 0.05 weight %, greater than 0.1 weight %, or greater than 0.5 weight %, based on a total weight of the personal care composition.

6. The personal care composition of claim 1, wherein the one or more sulfated polysaccharides comprise a linear sulfated polysaccharide comprising one or more sulphate groups.

7. The personal care composition of claim 1, wherein the one or more sulfated polysaccharides comprise a natural sulfated polysaccharide.

8. The personal care composition of claim 1, wherein the one or more sulfated polysaccharides comprise a synthetic sulfated polysaccharide.

9. The personal care composition of claim 1, wherein the one or more sulfated polysaccharides comprises one or more of carrageenan, keratan sulfate, chondroitin sulfate, dextran sulfate, dermatan sulfate, fucoidan, funoran, heparin, porphyran, or combinations thereof.

10. The personal care composition of claim 9, wherein the one or more sulfated polysaccharides comprises carrageenan, optionally, consists of carrageenan, further optionally, consists essentially of carrageenan.

11. The personal care composition of claim 10, wherein the carrageenan comprises one or more of kappa-carrageenan, iota-carrageenan, lambda-carrageenan, or combinations thereof, optionally, the carrageenan comprises kappa-carrageenan.

12. The personal care composition claim 1, wherein the personal care composition consists essentially of the carrier and the one or more sulfated polysaccharides, optionally, the personal care composition consists of the carrier and the one or more sulfated polysaccharides.

13. The personal care composition of claim 12, wherein the one or more sulfated polysaccharides consists essentially of carrageenan.

14. The personal care composition of claim 13, wherein the carrier is a solid carrier.

15. A method for preparing the personal care composition of claim 1, the method comprising contacting the one or more sulfated polysaccharides and the carrier with one another.

16. A method for treating, preventing, or reducing microbes on skin, the method comprising contacting the skin with any one of the personal care compositions of claim 14, optionally, the method further comprises increasing production of antimicrobial peptides in or on the skin.

17. The method of claim 16, wherein contacting the skin increases production of antimicrobial peptide biomarker LL-37 in or on the skin.

18. The method of claim 16, wherein contacting the skin increases an innate immune response.

19. A method for treating one or more dry skin conditions, the method comprising contacting the skin with any one of the personal care compositions of claim 14, the method further comprises increasing an amount of natural moisturizing factors in or on the skin.

20. The method of claim 19, wherein contacting the skin increases an amount of Caspace-14 in or on the skin.
